(19) United States
(12) Patent Application Publication
(10) Pub. No.: US 2026/0345491 A1
(43) Pub. Date: Nov. 5, 2026

(54) HYBRID NANOPARTICLES FOR TARGETED DELIVERY OF SENOLYTICS FOR THE TREATMENT OF OSTEOARTHRITIS

(71) Applicant: University of Kentucky Research Foundation, Lexington, KY (US)

(72) Inventors: Ramkumar Tiruvannamalai ANNAMALAI, Buffalo, NY (US); Sushant PRAJAPATI, Lexington, KY (US)

(21) Appl. No.: 19/663,541

(22) Filed: Apr. 30, 2026

Related U.S. Application Data
(60) Provisional application No. 63/797,487, filed on Apr. 30, 2025.

(51) Int. Cl. A61K 9/1271; A61K 31/245; A61K 31/352; A61K 31/506; A61K 47/42; A61K 47/46

(52) U.S. Cl. CPC A61K 9/1271; A61K 31/245; A61K 31/352; A61K 31/506; A61K 47/42; A61K 47/46

(57) ABSTRACT

A hybrid nanoparticle. The hybrid nanoparticle includes a synthetic lipid bilayer made of phosphatidylcholine, cholesterol, and a PEGylated phospholipid including a maleimide-functionalized PEG lipid and a biomembrane component derived from chondrocytes and incorporated into the synthetic lipid bilayer. The biomembrane component includes at least one SNARE protein configured to promote homotypic membrane fusion with chondrocytes and CD47 configured to inhibit phagocytic uptake. The hybrid nanoparticle further includes a collagen-binding targeting moiety covalently conjugated to the nanoparticle via the maleimide-functionalized PEG lipid and a therapeutic payload including a senolytic agent. The hybrid nanoparticle exhibits preferential internalization into chondrocytes relative to macrophages and increased intra-articular residence time relative to a corresponding nanoparticle lacking the biomembrane component. Related methods of making the hybrid nanoparticle and utilizing the hybrid nanoparticle for treatment of osteoarthritis are also provided.

### CROSS-REFERENCE TO RELATED APPLICATIONS

This application claims priority to U.S. Provisional Application 63/797487, filed Apr. 30, 2025, which is incorporated by reference herein in its entirety.

### TECHNICAL FIELD

Embodiments of the present disclosure generally relate to lipid-based nanoparticles. More specifically, embodiments of the present disclosure are related to hybrid nanoparticles formed as a combination of synthetic liposomes and cellular membranes loaded with a therapeutic payload comprising a senolytic agent as well as methods of treating osteoarthritis utilizing the hybrid nanoparticles.

### BACKGROUND

Osteoarthritis (OA) is a chronic, degenerative joint disorder characterized by the progressive deterioration of articular cartilage, subchondral bone remodeling, synovial inflammation, and formation of osteophytes. The condition most commonly affects weight-bearing joints such as the knees, hips, and spine, as well as the hands, and is a leading cause of pain and disability worldwide. The etiology of OA is multifactorial, involving mechanical stress, aging-related biochemical changes, genetic predisposition, and metabolic factors. Pathophysiologically, OA is associated with an imbalance between anabolic and catabolic processes in joint tissues, resulting in cartilage matrix degradation. Current treatment strategies for osteoarthritis are primarily symptomatic and include analgesics, nonsteroidal anti-inflammatory drugs (NSAIDs), serotonin-norepinephrine reuptake inhibitors (SNRI) such as Duloxetine (Cymbalta). And intra-articular injections for chronic pain, In advanced cases, surgical interventions such as joint replacement may be undertaken. However, these approaches do not adequately address the underlying disease progression, highlighting an ongoing need for improved therapeutic compositions and methods that can modify or halt the degenerative processes associated with OA.

### SUMMARY

As such, there is an unmet clinical need for the prevention and treatment of OA. The present hybrid nanoparticles of senolytic drugs in a lipid-nanoparticle formulation can address this unmet clinical need.

Embodiments of the present disclosure relate to a hybrid nanoparticle. The hybrid nanoparticle includes a synthetic lipid bilayer comprising phosphatidylcholine, cholesterol, and a PEGylated phospholipid including a maleimide-functionalized PEG lipid as well as a biomembrane component derived from chondrocytes and incorporated into the synthetic lipid bilayer. The biomembrane component includes (i) at least one SNARE protein configured to promote homotypic membrane fusion with chondrocytes; and (ii) CD47 configured to inhibit phagocytic uptake. The hybrid nanoparticle also includes a collagen-binding targeting moiety covalently conjugated to the nanoparticle via the maleimide-functionalized PEG lipid and a therapeutic payload comprising a senolytic agent. Further, the hybrid nanoparticle exhibits preferential internalization into chondrocytes relative to macrophages and increased intra-articular residence time relative to a corresponding nanoparticle lacking the biomembrane component.

Embodiments of the present disclosure also relate to a method of making a hybrid nanoparticle. The method includes (a) extracting a biomembrane from chondrocytes, the biomembrane comprising SNARE proteins and CD47; (b) combining the biomembrane with a synthetic lipid mixture comprising phosphatidylcholine, cholesterol, and a PEGylated maleimide lipid; (c) extruding the combination through a membrane having a pore size of about 100 nm to form hybrid nanoparticles; and (d) conjugating a collagen-binding antibody to the hybrid nanoparticles via thiol–maleimide chemistry.

Embodiments of the present disclosure also relate to a method of treating osteoarthritis in a subject. The method includes administering intra-articularly to the subject the hybrid nanoparticle of claim 1 in an amount effective to(a) selectively eliminate senescent chondrocytes; (b) reduce inflammatory signaling associated with senescence-associated secretory phenotype (SASP); and (c) inhibit progression of cartilage degeneration.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals.

FIG. 1 is a schematic illustration of a hybrid nanoparticle in accordance with one or more embodiments of the present disclosure.

FIG. 2 is a schematic illustration of targeted removal of senescent cells in osteoarthritis in accordance with one or more embodiments of the present disclosure.

FIG. 3A is an electron micrograph of hybrid nanoparticles in accordance with one or more embodiments of the present disclosure.

FIG. 3B is an electron micrograph of internalization of hybrid nanoparticles by chondrocytes in accordance with one or more embodiments of the present disclosure.

FIG. 3C is a graph of uptake of hybrid nanoparticles and comparative particles by chondrocytes in accordance with one or more embodiments of the present disclosure.

FIG. 3D is a graph of uptake of hybrid nanoparticles and comparative particles by macrophages in accordance with one or more embodiments of the present disclosure.

FIG. 4 is flowchart overview of types of senescence in accordance with one or more embodiments of the present disclosure.

FIG. 5 is a schematic illustration of senolytic action to restore cells and/or tissue in accordance with one or more embodiments of the present disclosure.

FIG. 6 is schematic illustration and electron micrographs of the formation of a hybrid nanoparticles in accordance with one or more embodiments of the present disclosure in accordance with one or more embodiments of the present disclosure.

FIG. 7A is a graph of size homogeneity of synthetic immune-liposome with and without senolytic agent loading in accordance with one or more embodiments of the present disclosure.

FIG. 7B is a graph of size homogeneity of synthetic immune-liposomes and biomembranes in accordance with one or more embodiments of the present disclosure.

FIG. 7C is a graph of size homogeneity and stability of hybrid nanoparticles over 28 days in accordance with one or more embodiments of the present disclosure.

FIG. 8 are images of proliferative chondrocytes and lab-induced senescent chondrocyte in accordance with one or more embodiments of the present disclosure s.

FIG. 9A is a graph of toxicity of dasatinib to proliferative macrophages in accordance with one or more embodiments of the present disclosure.

FIG. 9B is a graph of toxicity of quercetin to proliferative macrophages in accordance with one or more embodiments of the present disclosure.

FIG. 10A is a graph of toxicity of dasatinib to proliferative chondrocytes in accordance with one or more embodiments of the present disclosure.

FIG. 10B is a graph of toxicity of quercetin to proliferative chondrocytes in accordance with one or more embodiments of the present disclosure.

FIG. 11A is a graph of toxicity of immune-liposomes as a drug carrier for dasatinib to proliferative macrophages in accordance with one or more embodiments of the present disclosure.

FIG. 11B is a graph of toxicity of immune-liposomes as a drug carrier for quercetin to proliferative macrophages in accordance with one or more embodiments of the present disclosure.

FIG. 12A is a graph of toxicity of immune-liposomes as a drug carrier for dasatinib to proliferative chondrocytes in accordance with one or more embodiments of the present disclosure.

FIG. 12B is a graph of toxicity of immune-liposomes as a drug carrier for quercetin to proliferative chondrocytes in accordance with one or more embodiments of the present disclosure.

FIG. 13A is a graph of decrease in metabolism of senescent chondrocytes over 7 days when exposed to various concentrations of dasatinib in accordance with one or more embodiments of the present disclosure.

FIG. 13B is a graph of decrease in metabolism of senescent chondrocytes over 7 days when exposed to various concentrations of quercetin in accordance with one or more embodiments of the present disclosure.

FIG. 14 is a graph illustrating the uptake of the hybrid nanoparticles and comparative particles into chondrocytes in accordance with one or more embodiments of the present disclosure.

FIG. 15 is a graph illustrating the uptake of the hybrid nanoparticles and comparative particles into macrophages in accordance with one or more embodiments of the present disclosure.

### DETAILED DESCRIPTION

Embodiments of the present disclosure generally relate to lipid-based hybrid nanoparticle loaded with disease-modifying drugs, specifically senolytics, that selectively target eroding cartilage to decrease the burden of senescent cells in osteoarthritis (OA) patients. The hybrid nanoparticles are formulated and structured to increase the intra-articular residence time of these drugs and provide a therapeutic dosage through a unique and hybrid formulation. Specifically, the hybrid nanoparticles are a product of synthetic liposomes combined with cellular membranes or a derivative or portion thereof to effectively deliver the senolytics to the target tissue.

Senolytics therapies are an emerging market, and several age-related diseases could benefit from the hybrid nanoparticles of the present disclosure. Some of the diseases include osteoarthritis, age-related macular degeneration (AMD), diabetic edema, cardiovascular disease, Alzheimer's disease, Parkinson's disease, cancer, diabetes, chronic obstructive pulmonary disease (COPD), and kidney disease. However, for conciseness and clarity the present disclosure focuses on osteoarthritis with it expressly noted and understood that the same techniques and hybrid nanoparticles may similarly be levered in other noted disease applications. Such additional disease applications are expressly noted as being applicable to the present disclosure.

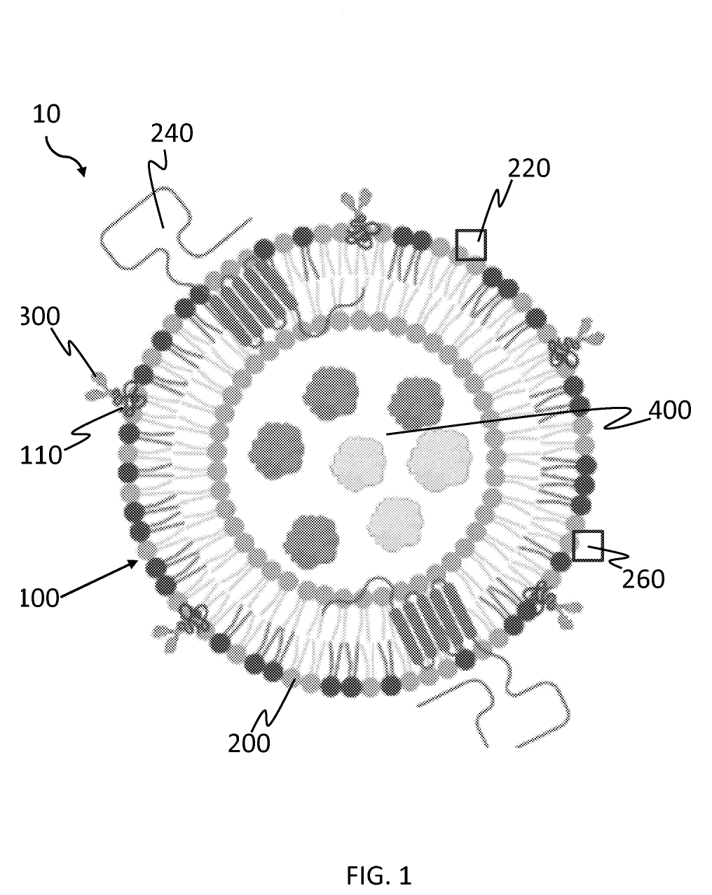

In accordance with embodiments of a hybrid nanoparticle 10 and with reference to FIG. 1, the hybrid nanoparticle 10 comprises a plurality of layers. The hybrid nanoparticle 10 comprises a synthetic lipid bilayer 100 comprising phosphatidylcholine, cholesterol, and a PEGylated phospholipid including a maleimide-functionalized PEG lipid 110. The hybrid nanoparticle 10 further comprises a biomembrane component 200 derived from chondrocytes and incorporated into the synthetic lipid bilayer 100. The biomembrane component 200 comprises at least one SNARE protein 220 (soluble N-ethylmaleimide-sensitive factor attachment protein receptors) configured to promote homotypic membrane fusion with chondrocytes and CD47 240 configured to inhibit phagocytic uptake. The hybrid nanoparticle 10 additionally comprises a collagen-binding targeting moiety 300 covalently conjugated to the hybrid nanoparticle 10 via the maleimide-functionalized PEG lipid 110. Finally, the hybrid nanoparticle 10 comprises a therapeutic payload 400. The therapeutic payload comprises one or more senolytic agents. Accordingly, the hybrid nanoparticle 10 exhibits preferential internalization into chondrocytes relative to macrophages and increased intra-articular residence time relative to a corresponding nanoparticle lacking the biomembrane component.

Having generally described the various components of the hybrid nanoparticle 10, each component and synthesis mechanisms will be described in further detail.

In accordance with one or more embodiments, the hybrid nanoparticle 10 includes a synthetic lipid bilayer 100. The synthetic lipid bilayer 100 comprises phosphatidylcholine, cholesterol, and a PEGylated phospholipid including a maleimide-functionalized PEG lipid 110. Specifically, in accordance with one or more embodiments, the synthetic lipid bilayer 100 may be formed from a mixture of synthetic lipids 1,2-Dioleoyl-sn-glycero-3-phosphocholine (DOPC), cholesterol, 1,2-distearoyl-sn-glycero-3-phosphoethanolamine-N-[amino(polyethylene glycol)-2000] (DSPE-PEG-2K), and DSPE-PEG-2K-maleimide. In one or more embodiments, the DOPC is provided at 5.2 mM, cholesterol at 4.5 mM, DSPE-PEG-2K at 0.3 mM, and DSPE-PEG-2K-maleimide at 15 µM.

In accordance with one or more embodiments, the synthetic lipid bilayer 100 may then be modified with a biomembrane component 200 derived from chondrocytes which are incorporated into the synthetic lipid bilayer 100. Specifically, the synthetic lipid bilayer 100 may have phospholipid bilayers with transmembrane proteins incorporated into the synthetic lipid bilayer 100. In accordance with various embodiments, the transmembrane proteins in the phospholipid bilayers incorporated into the synthetic lipid bilayer 100 may include at least one SNARE protein 220 and/or CD47 240 and/or cadherins 260.

In accordance with one or more embodiments, the biomembrane component 200 incorporated into the synthetic lipid bilayer 100 compromises at least one SNARE protein 220 configured to promote homotypic membrane fusion with chondrocytes. As appreciated by one skilled in the art, a primary role of SNARE proteins 220 is to mediate the fusion of vesicles with a target membrane; this notably mediates exocytosis, but can also mediate the fusion of vesicles with membrane-bound compartments such as a lysosome. Specifically, the SNARE protein 220 promotes localizing and fusion of the hybrid nanoparticle 10 with chondrocytes of a subject to be treated. In one or more specific embodiments, the SNARE protein may be VAMP-3 (vesicle-associated membrane protein 3) encoded by the human VAMP3 gene. Additional or alternative SNARE proteins 220 known to those skilled in the art may additionally or alternatively be utilized.

In accordance with one or more embodiments, the biomembrane component 200 incorporated into the synthetic lipid bilayer 100 compromises CD47 240. The CD47 240 is provided as part of the biomembrane component 200 to inhibit phagocytic uptake. Specifically, CD47 240 is a transmembrane protein known as a "don't eat me" signal and is expressed on normal cells to prevent phagocytosis by macrophages. That is, CD47 240 binds to SIRPα receptors on macrophages, triggering an inhibitory signal that stops them from eating healthy cells. Inclusion of CD47 240 in the biomembrane component 200 alleviates undesirable phagocytosis by macrophages of the hybrid nanoparticle 10 allowing the hybrid nanoparticle 10 to remain at the site of eroding cartilage to deliver the senolytic agent.

In accordance with one or more embodiments, the hybrid nanoparticle 10 additionally includes a collagen-binding targeting moiety 300 covalently conjugated to the hybrid nanoparticle 10 via the maleimide-functionalized PEG lipid 110. Specifically, the hybrid nanoparticle bmay include a collagen-binding targeting moiety 300 to target collagen cells in a subject to be treated with the hybrid nanoparticles 10. In accordance with one or more embodiments, the collagen-binding targeting moiety 300 may be thiolated collagen-II antibodies which are conjugated to the hybrid nanoparticle 10 through maleimide chemistry. It is noted that collagen II antibodies (anti-CII) are specialized antibodies for detecting and targeting type II collagen, primarily found in cartilage, bone, and connective tissues. Collagen-II antibodies specifically target the Collagen II protein or alpha-1(II) chain.

In accordance with one or more embodiments, the hybrid nanoparticle 10 additionally includes a therapeutic payload 400. Specifically, the therapeutic payload 400 is disposed at the interior of the hybrid nanoparticle 10 allowing for delivery to a target site. In one or more embodiments, the hybrid nanoparticle 10 degrades within the subject allowing for the therapeutic payload 400 to be delivered.

In accordance with one or more embodiments, the therapeutic payload 400 includes a senolytic agent. Specifically, senolytic drugs or senolytic agents are a class of small molecules and natural compounds that selectively induce death in senescent ("zombie") cells. It will be appreciated that senescent cells are damaged cells that stop dividing but do not die, becoming "zombie" cells that linger, secrete inflammatory factors (SASP), and harm surrounding tissue. Senescent cells accumulate with age and drive chronic diseases.

In accordance with one or more embodiments, the senolytic agent provided as at least a portion of the therapeutic payload 400 includes dasatinib and quercetin. For example, a combination of Dasatinib, a tyrosine kinase inhibitor, may be provided at 1-10 µM and Quercetin, a flavonoid and antioxidant, may be provided at 10-1000 µM to target senescent cells. These senolytic drugs prevent or lessen osteoarthritis by transiently disabling pro-survival pathways, protecting senescent cells from their apoptotic signals. Additional or alternative senolytic agents known to those skilled in the art may additionally or alternatively be utilized as part of the therapeutic payload 400 with dasatinib and quercetin provided as examples.

In accordance with one or more embodiments, a sirtuin activator may additionally be provided as at least a portion of the therapeutic payload 400. Sirtuin (SIRT) activators are natural compounds and synthetic molecules that boost the activity of sirtuins which are proteins regulating metabolic health, inflammation, and cellular aging. Specifically, sirtuins regulate critical processes, including inflammation, DNA repair, and energy homeostasis, often acting as "longevity genes" by delaying age-related diseases and supporting mitochondrial health. In one or more embodiments, the sirtuin activator provided in the therapeutic payload 400 comprises MDL-800 which may be provided at 10-100 µM. MDL-800 is a sirtuin-6 activator which also improves the genomic stability of senescent cells and eliminates chronic low-level inflammation that results in osteoarthritis. Additional or alternative sirtuin activators known to those skilled in the art may additionally or alternatively be utilized.

Previous attempts in the art to generate synthetic lipid-based vesicles for drug delivery lack the ability to target specific tissues and evade the immune system. These two properties are essential for senolytic and senomorphic drugs to exhibit disease-modifying effects in OA joints while minimizing off-target effects. The hybrid nanoparticle 10 formulation is unique in that it combines synthetic and biological components (synthetic lipids and membrane proteins) that harness physical tenets and biological pathways to deliver the cargo to the OA knee actively. In particular, the SNARE proteins (chondrocyte specific) incorporated in the hybrid nanoparticle 10 formulation can induce membrane fusion with exposed chondrocytes in OA through homotypic targeting mechanisms. Further, with another type of transmembrane protein (CD47), the hybrid nanoparticles 10 are protected from the reticuloendothelial system (RES), leading to improved pharmacokinetic properties including enhanced drug half-life and residence time in the knee or other arthritic joint.

In accordance with one or more embodiments, the hybrid nanoparticle 10 further includes fluorophores to allow visualization of the hybrid nanoparticles 10. Specifically, as understood by one skilled in the art, a fluorophore is a fluorescent chemical compound that absorbs light energy (photons) at a specific excitation wavelength and re-emits it at a longer, lower-energy wavelength resulting in fluorescence. The fluorescence may be leveraged to label the hybrid nanoparticles 10 allowing for tracking and visualization of the hybrid nanoparticles 10. Non-limiting examples of fluorophores which may be utilized include fluorescein isothiocyanate (FITC), Dil, DiR, or DiD. For example, FITC is a green-fluorescent dye (excitation/emission 495/519 nm) used for antibody conjugation and DiI (excitation/emission 549/565 nm), DiR (excitation/emission 750-760/775-785 nm), and DiD (excitation/emission 644/665 nm) are lipophilic, carbocyanine membrane tracers that fluoresce brightly only when bound to lipid bilayers, appearing orange-red (DiI), red (DiD), and near-infrared (DiR) allowing for cell tracking and imaging.

In accordance with one or more embodiments, the hybrid nanoparticles 10 comprises a substantially homogenous size distribution. For example, in one or more embodiments, the hybrid nanoparticles 10 have an average diameter of 100 to 200 nm and a polydispersivity index of less than 0.3. In accordance with various further embodiments, the hybrid nanoparticles 10 have an average diameter of 100 to 175 nm, 125 to 200 nm, 125 to 175 nm, 135 to 165 nm, or 145 to 155 nm and a polydispersivity index of less than 0.29, less than 0.28, less than 0.27, less than 0.26, 0.1 to 0.3, 0.15 to 0.3, or 0.2 to 0.3. In accordance with one or more specific embodiments, the hybrid nanoparticles 10 have an average diameter of 140-150 nm (targeted to 145.6 nm) and a polydispersivity index of approximately 0.25.

In accordance with one or more embodiments, the hybrid nanoparticles 10 comprise a unilamellar structure. It will be appreciated that a unilamellar structure is a spherical liposome or vesicle consisting of a single phospholipid bilayer membrane enclosing an aqueous core which mimic cell membranes.

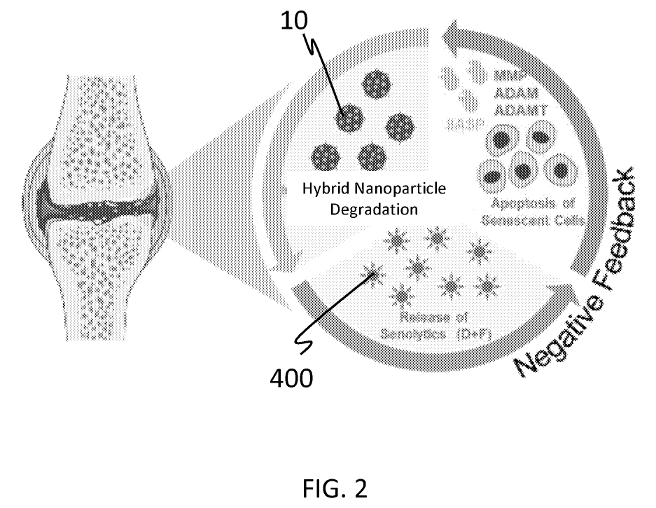

In accordance with one or more embodiments and with reference to FIG. 2, osteoarthritis may be treated in a subject with targeted removal of senescent cells with administration of the hybrid nanoparticle 10. Specifically, in one or more embodiments, a method of treating osteoarthritis in a subject is provided. The method comprises administering intra-articularly to the subject the hybrid nanoparticle 10 in an amount effective to selectively eliminate senescent chondrocytes, reduce inflammatory signaling associated with senescence-associated secretory phenotype (SASP), and inhibit progression of cartilage degeneration. As illustrated in FIG. 2, the hybrid nanoparticles 10 degrade releasing the therapeutic payload 400 including one or more senolytic agents which trigger apoptosis of senescent cells reducing inflammatory factors which contribute to cartilage degradation in osteoarthritis.

To demonstrate the utility of the hybrid nanoparticle 10, example hybrid nanoparticles 10 were generated. The method of making the hybrid nanoparticles 10 as well as further aspects of the hybrid nanoparticles 10 are disclosed in conjunction with discussion of the example hybrid nanoparticles 10.

Hybrid nanoparticles were generated by combining a mixture of synthetic lipids including 1,2-Dioleoyl-sn-glycero-3-phosphocholine (DOPC), cholesterol, 1,2-distearoyl-sn- glycero-3-phosphoethanolamine-N-[amino(polyethylene glycol)-2000] (DSPE-PEG-2K), DSPE-PEG-2K-maleimide, and phospholipid bilayers with transmembrane proteins (including v-SNAREs (VAMP-3), CD47, and Cadherins). The phospholipid bilayers with transmembrane proteins were extracted from live 1-5 million chondrocytes, specifically ATDC5 cells. In the generated hybrid nanoparticles DOPC is present at 5.2 mM, cholesterol at 4.5 mM, DSPE-PEG-2K at 0.3 mM, DSPE-PEG-2K-maleimide at 15 µM. Subsequently, the mixture was extruded through a 100 nm polycarbonate membrane to generate the hybrid nanoparticles 10.

In accordance with one or more embodiments, a method of making the hybrid nanoparticle 10 is provided. The method comprises extracting a biomembrane from chondrocytes, the biomembrane comprising SNARE proteins and CD47, combining the biomembrane with a synthetic lipid mixture comprising phosphatidylcholine, cholesterol, and a PEGylated maleimide lipid, extruding the combination through a membrane having a pore size of about 100 nm to form hybrid nanoparticles, and conjugating a collagen-binding antibody to the hybrid nanoparticles via thiol–maleimide chemistry.

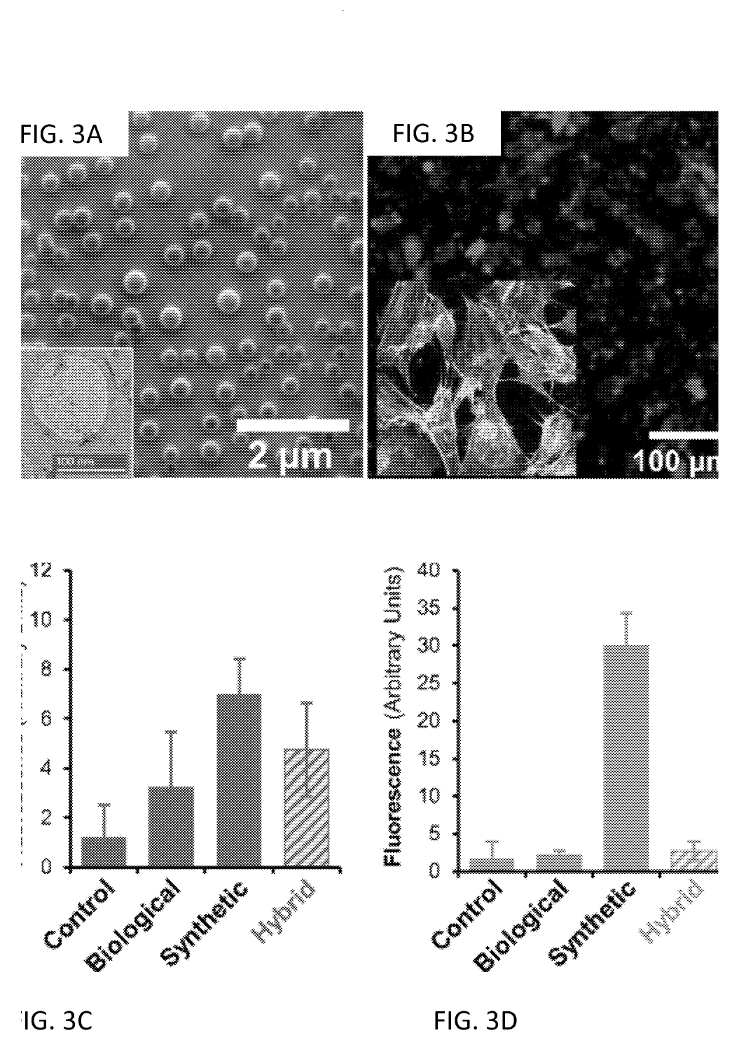

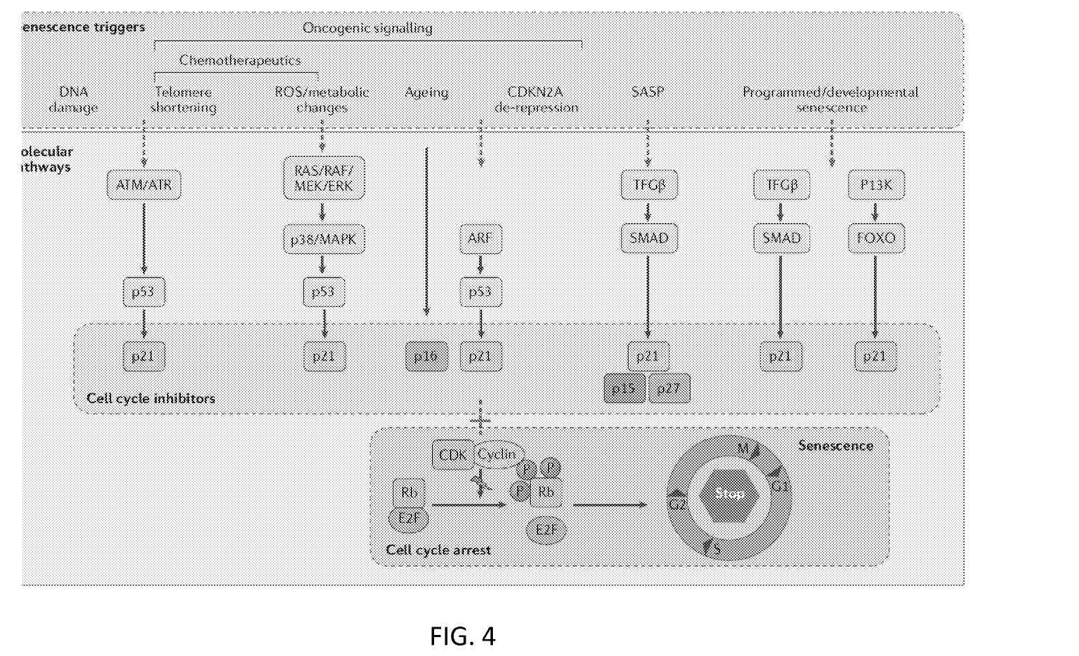

The formed hybrid nanoparticles 10 are depicted in FIG. 3A and 3B. Specifically, FIG. 3A illustrates the hybrid nanoparticles 10. Further, the inset of FIG. 3A highlights the unilamellar structure of the generated hybrid nanoparticles 10 with a cross-section in a transmission electron microscope. FIG. 3B provides an illustration of the internalization by chondrocytes of the hybrid nanoparticles 10 with the hybrid nanoparticles 10 integrated into the actin cytoskeleton of chondrocytes.

The hybrid nanoparticles 10 were stable in aqueous solutions for weeks without aggregation or precipitation. Further, with reference to FIG. 3C, the hybrid nanoparticles 10 were internalized by chondrocytes (parenchymal cells in cartilage tissue) at the same rate as nanoparticles generated from the mixture of synthetic lipids without the phospholipid bilayers with transmembrane proteins (labeled as “synthetic” in FIGS. 3C and 3D). However, the hybrid nanoparticles 10 were overlooked by the phagocytic macrophages as illustrated in FIG. 3D. The selective internalization by the chondrocytes and evasion by the phagocytic cells can be attributed to the presence of specialized proteins on the surface of the hybrid nanoparticles 10. The biomembrane was also tested alone (labeled as “biological” in FIGS. 3C and 3D). Control samples without introduction of any additional components were also completed (labeled as “control” in FIGS. 3C and 3D).

The hybrid nanoparticles 10 target senescent cells in treating osteoarthritis. FIG. 4 shows an overview of types of senescence: Programmed/developmental, Replicative, and Stress-Induced Premature Senescence (SIPS). Pathological senescence involves metabolically active cells that undergo cell cycle arrest and can cause inflammation and affect non-senescent cells. Senescence can be caused by factors such as reactive oxygen species (ROS), DNA damage, and SASP. Senescence is mainly identified by the over expression of the biomarkers p16^{INK4a} , p21^{cip1} and SASP. These senescent cells cause chronic inflammation by secreting inflammatory factors collectively known as SASP – which leads to progression of the disease. Accordingly, it is believed that eliminating senescent cells using a new class of drug called senolytics may have disease modifying effects in OA joints. However, these drugs have very short half-lives. Furthermore, the safety of oral administration has been questioned, and the off-target effects of these pro-apoptotic molecules constitute a significant concern. The present disclosure is not looking into non-pathological senescence which happens during embryonic stages for tissue remodeling or homeostasis maintenance.

The present disclosure focuses on senescence in pathological scenarios, where metabolically active cells undergo cell cycle arrest and accumulate in the tissue and cause chronic inflammation by secreting inflammatory factors collectively known as SASP. This is contrasted with non-pathological senescence, which is not the focus of the present disclosure, which happens during embryonic stages for tissue remodeling or homeostasis maintenance. The primary triggers that cause the senescence could be DNA damage or ROS production or SASP or ageing. These senescent cells can be identified through specific biomarkers and they vary with the type of triggers but p16 and p21 has been commonly implicated in ageing related phenotype that we are interested in. The strategy of the present disclosure is to specifically eliminate these phenotype in OA. The present disclosure aims to introduce senolytics which are a class of drugs that selectively eliminate these cells.

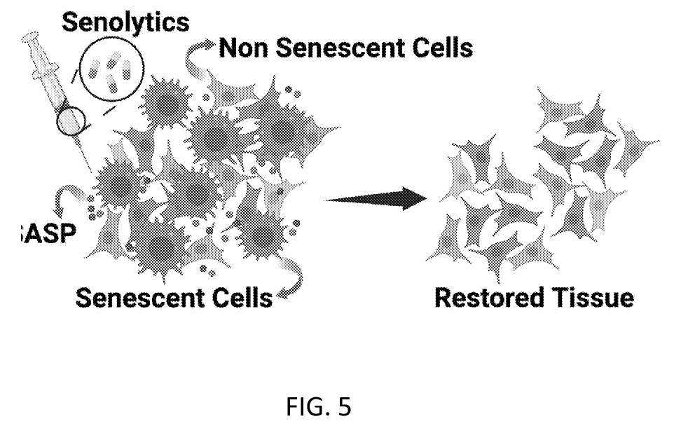

As previously noted, administration of the hybrid nanoparticles 10 is at least in part to eliminate senescent cells. FIG. 5 shows an overview of senolytic action to restore cells and/or tissue. Senolytics are a class of drugs that have been shown to selectively eliminate senescent cells by inhibiting the pro-survival networks which protects them from their own apoptotic factors. These senolytics may include dasatinib, which functions as a Tyrosine Kinase-inhibitor (e.g. BCR-ABL, SRC, c-KIT, ephrin A receptor) as well as Quercetin which is a plant flavonol and can function as a PI3K and Serpines Inhibitor. It is believed that the combination of dasatinib and Quercetin selectively eliminate senescent cells by transiently disabling pro-survival networks that defend them against their own apoptotic environment. As illustrated in FIG. 5. The selective removal of senescent cells concurrently eliminates SASP allowing for the underling tissue to be restored providing relief for an osteoarthritic patient.

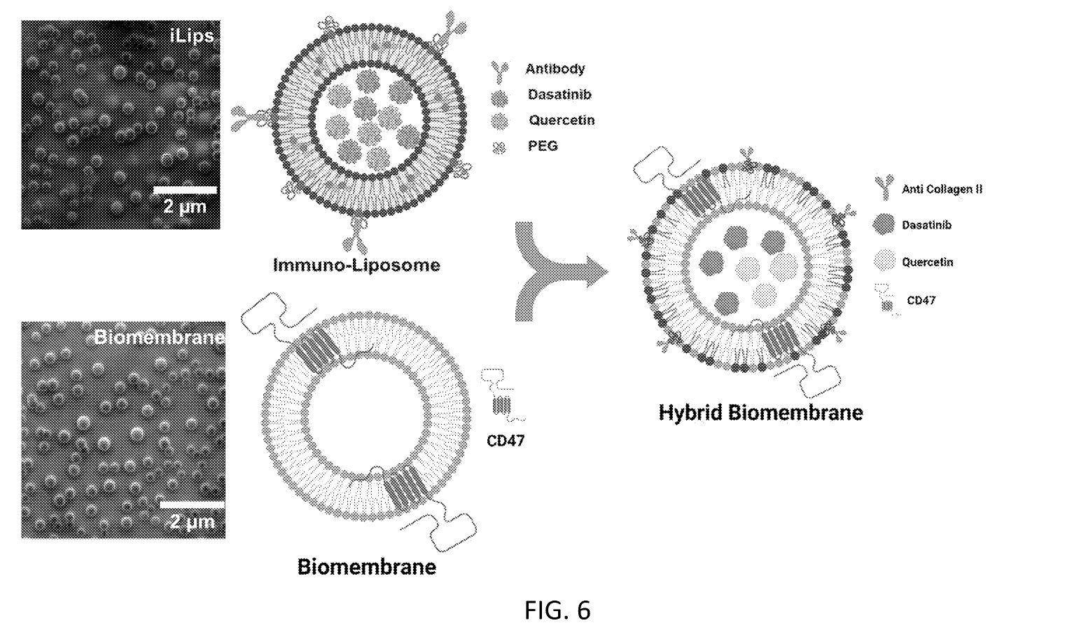

The hybrid nanoparticles 10 include an immune-evasive drug (senolytics) carrier that selectively target eroding cartilage to decrease the burden of senescent cells in OA. By transiently promoting apoptosis of senescent cells in exposed cartilage, the hybrid nanoparticles 10 of the present disclosure can prevent chronic inflammation and eliminate OA development and progression. The hybrid nanoparticles 10 are the product of immuno-liposomes (iLips) (e.g. Anti-ColI AB) and chondrocyte biomembranes (biomembrane) (e.g. CD47+Homotypic targeting). FIG. 6 provides an overview of the combination. The immune-liposomes are made through extruding a mixture of lipids (Phosphatidylcholine, cholesterol, DSPE-PEG) which generates the synthetic lipid bilayer 100. Similarly, the biomembranes were made through extrusion of chondrocytes that have the advantage of having “do not eat me signal” with the transmembrane protein CD47 in their membranes and membrane proteins which have the potential to target the cartilage via homotypic mechanisms to provide the biomembrane component 200. Combining these two ingredients to create hybrid nanoparticles 10 (Hybrid Biomembrane) have the ability to not only target the damaged cartilage but also, they are also immune evasive.

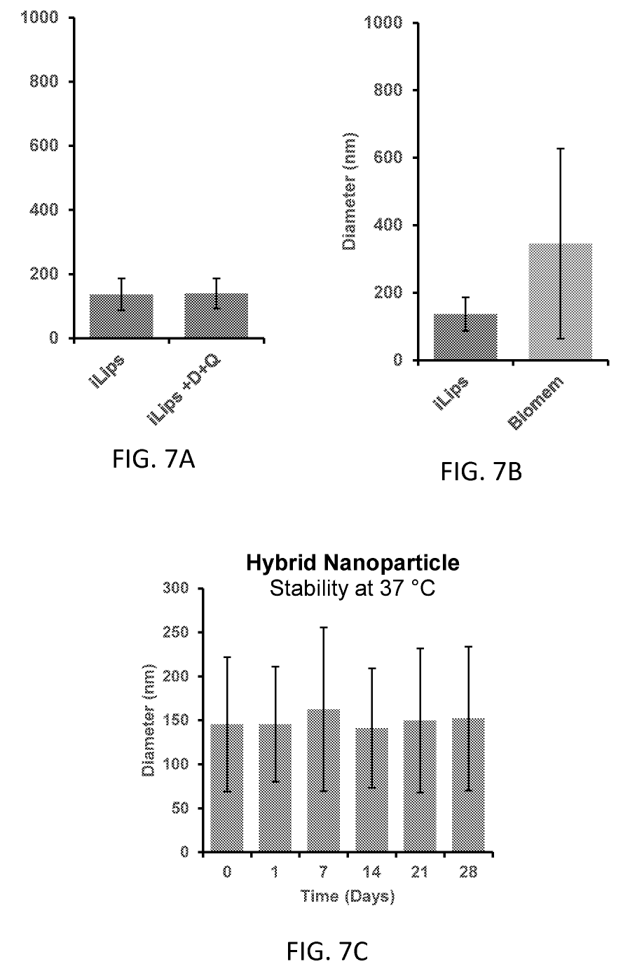

The hybrid nanoparticles 10 exhibit uniform size and stability. With reference to FIGS. 7A, 7B, and 7C, each of the immune-Liposome (iLips), biomembrane (Biomem), and hybrid nanoparticle 10 exhibit stability and size homogeneity. Specifically, with reference to FIGS. 6 and 7A, the synthetic Immuno-Liposome demonstrates a homogeneous size distribution and shown to be stable for weeks with or without the inclusion of senolytic agents with comparison of the synthetic Immuno-Liposome alone and in combination with dasatinib and quercetin (iLips +D+Q). The biomembranes exhibited homogenous size distribution after synthesis as illustrated in FIGS. 6 and 7B but became unstable over time. When the synthetic Immuno-Liposome and biomembranes are combined together to create the hybrid nanoparticles 10, they form a homogeneous particles that demonstrates stability for weeks as illustrated in FIG. 7C. Specifically, FIG. 7C shows stability is maintained for at least 28 days (4 weeks).

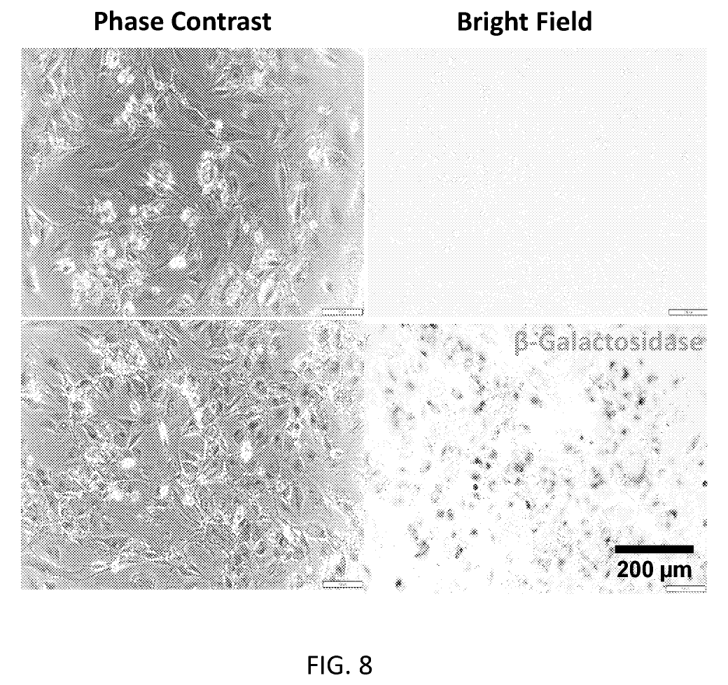

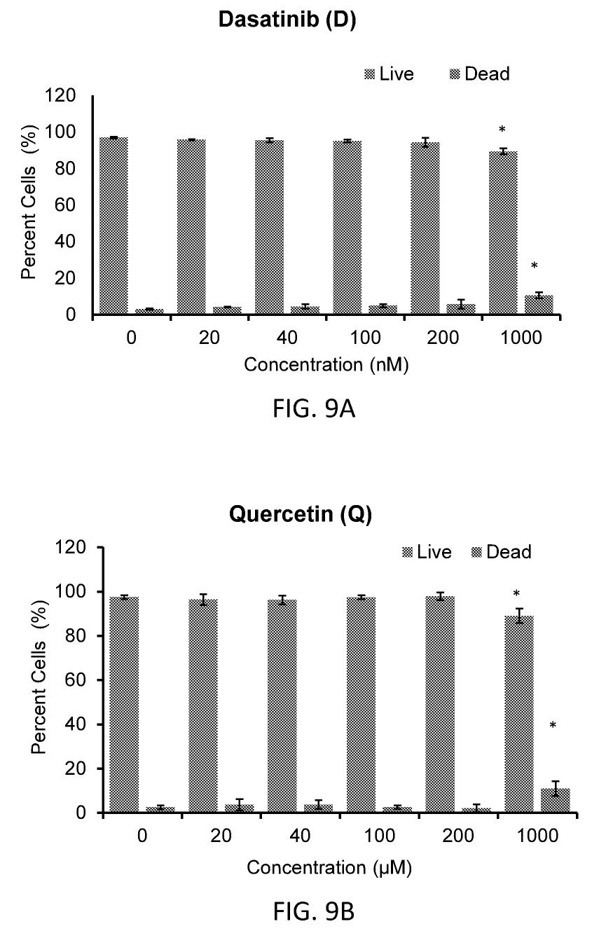

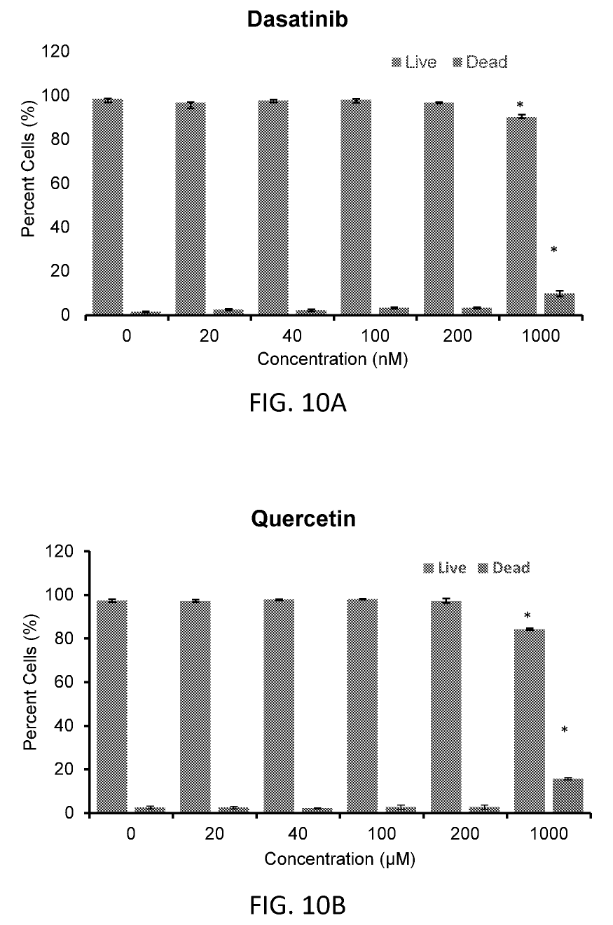

It was then tested how hybrid nanoparticles 10 behave as well as the effect on surrounding cells in in vitro conditions. Senescent cells were first created by treating healthy chondrocyte with bleomycin. FIG. 8 shows that these senescent cells overexpress β-gal. The effect of inclusion of dasatinib and quercetin in the hybrid nanoparticles 10 on macrophages and chondrocytes was tested. Specifically, with reference to FIGS. 9A and 9B , it may be shown that dasatinib and quercetin respectively are minimally toxic to proliferative macrophages. It was found that the proliferative macrophages were able to tolerate the dasatinib and quercetin ranging from 0 nM to 1000 nM (1 µM) for dasatinib and 0 µM to 1000 µM (1 mM) for quercetin. Similarly, with reference to FIGS. 10A and 10B, the chondrocytes were able to tolerate the dasatinib and quercetin ranging from 0 nM to 1000 nM (1 µM) for dasatinib and 0 µM to 1000 µM (1 mM) for quercetin respectively.

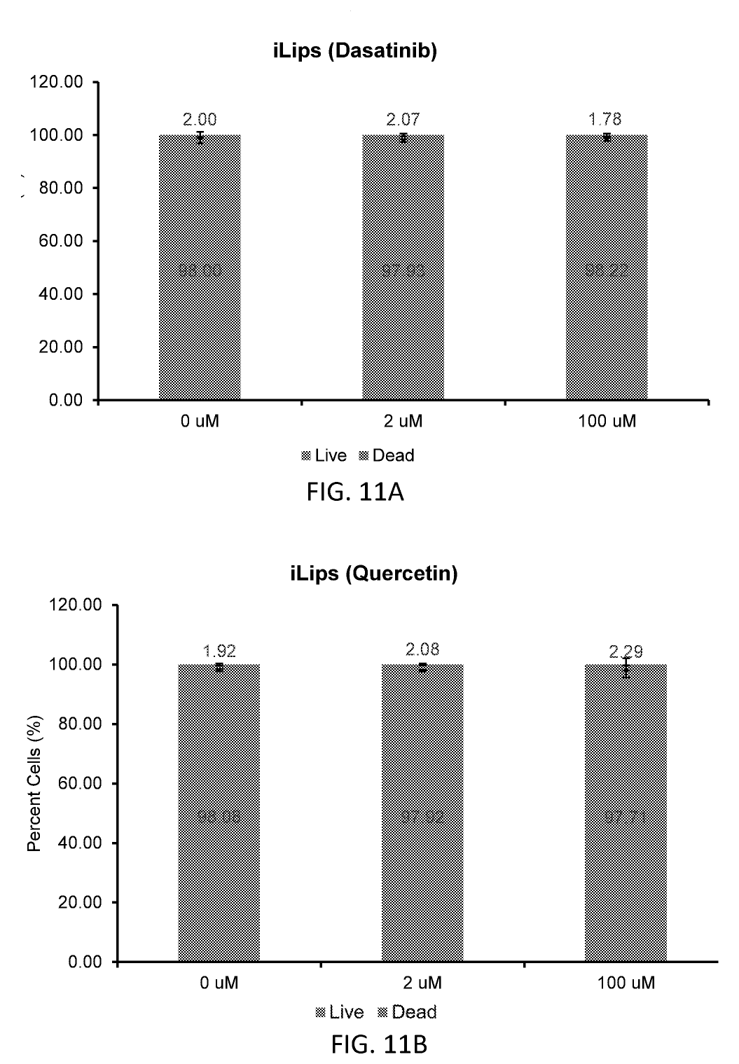

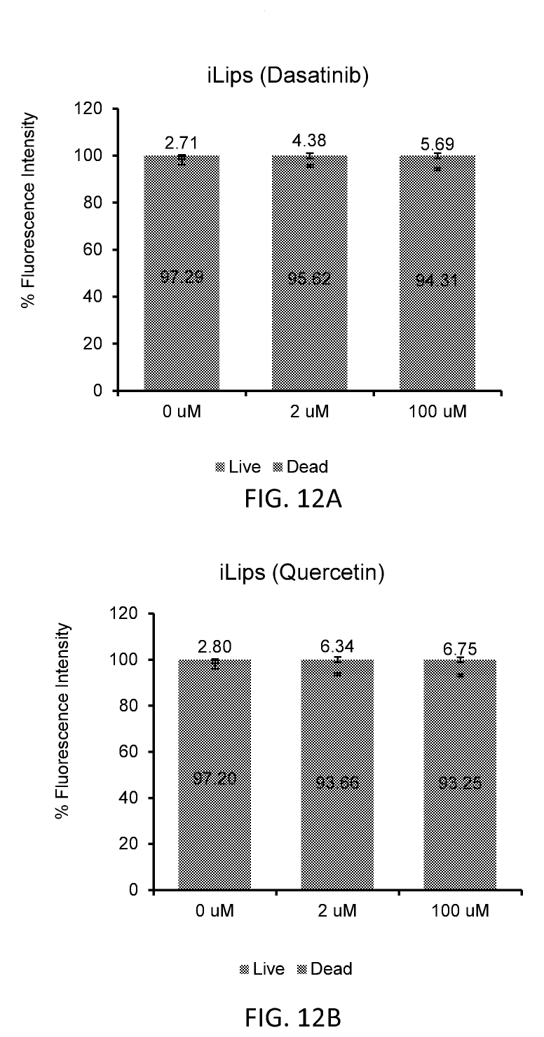

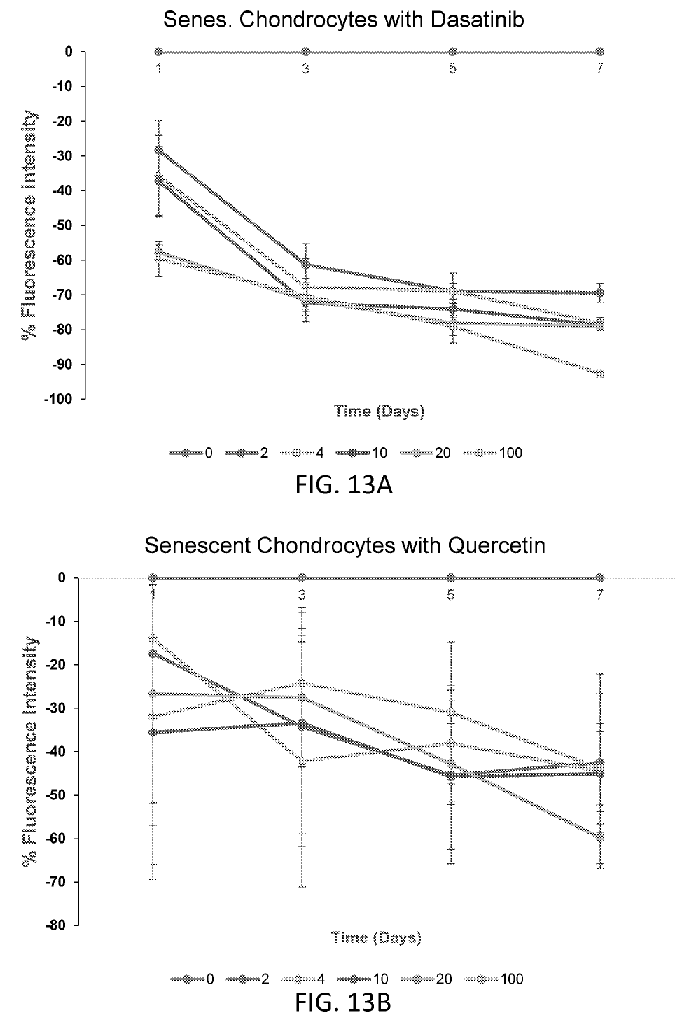

Further, FIGS. 11A and 11B shows that high drug payload for dasatinib and quercetin respectively provided in a synthetic immuno-liposome was minimally toxic to proliferating macrophages. Similarly, FIGS. 12A and 12B shows that high drug payload for dasatinib and quercetin respectively was minimally toxic to proliferating chondrocytes. However, as seen in FIGS. 13A and 13B, there was a desirable decreased metabolism of senescent cells even at low concentrations of senolytic. Specifically, a review of FIG. 13A illustrating the metabolism of senescent chondrocytes exposed to dasatinib and FIG. 13B illustrating the metabolism of senescent chondrocytes exposed to quercetin both display a reduction in florescence intensity over a period of 7 days for concentrations of senolytic ranging from 0 to 100 µM. The 0 µM samples retained full fluorescence intensity and each sample from 2 to 100 µM for both dasatinib and quercetin demonstrated a marked decrease in fluorescence intensity over the 7 days.

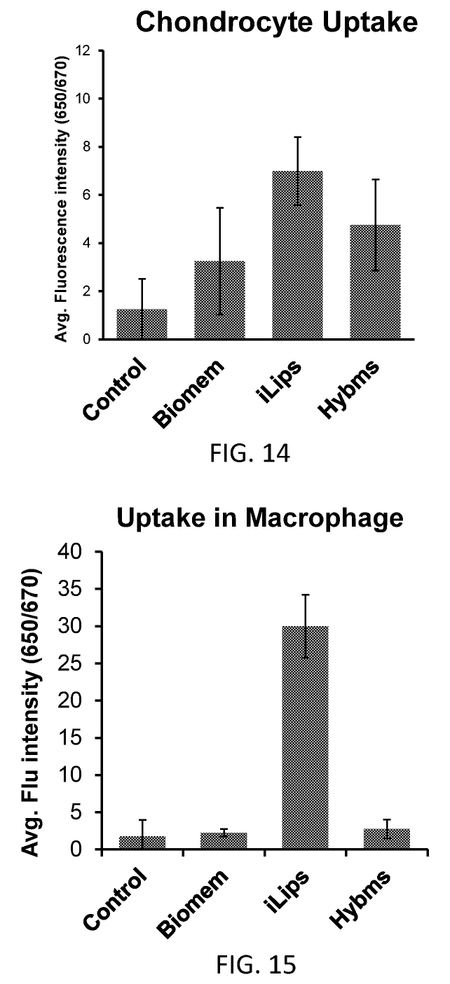

It was then quantified how efficiently the hybrid nanoparticles 10 (Hybms) were internalized by chondrocytes in comparison to synthetic immune-liposomes (iLips) and biomembranes (Biomem). FIG. 14 shows that the hybrid nanoparticles 10 are readily internalized by chondrocytes. The biomembranes are not particularly stable and tended to aggregate more, thereby compromising internalization. However, when combined with the immune-liposomes, the resulting hybrid nanoparticles 10 are stable with enhanced internalization. This shows that the combination of the synthetic liposomes with chondrocyte membrane in the hybrid nanoparticles 10 are stable, tunable, and importantly evade phagocytic cells due to presence of CD47. Similarly, FIG. 15 shows that the hybrid nanoparticles 10 evade the phagocytic macrophages. Accordingly, the present disclosure shows that the hybrid nanoparticles 10 are highly stable and homogenous. They are also readily internalized by chondrocytes and able to deliver therapeutics, while evading phagocytic macrophages.

Having described various embodiments, it should be understood that the various aspects of the hybrid nanoparticles and methods for making the same and using the same in treatment of osteoarthritis in a subject are described and such aspects may be utilized in conjunction with various other aspects.

In a first aspect, the disclosure provides a hybrid nanoparticle. The hybrid nanoparticle comprises (a) a synthetic lipid bilayer comprising phosphatidylcholine, cholesterol, and a PEGylated phospholipid including a maleimide-functionalized PEG lipid; (b) a biomembrane component derived from chondrocytes and incorporated into the synthetic lipid bilayer, the biomembrane component comprising: (i) at least one SNARE protein configured to promote homotypic membrane fusion with chondrocytes; and (ii) CD47 configured to inhibit phagocytic uptake; (c) a collagen-binding targeting moiety covalently conjugated to the nanoparticle via the maleimide-functionalized PEG lipid; and (d) a therapeutic payload comprising a senolytic agent, wherein the hybrid nanoparticle exhibits: (i) preferential internalization into chondrocytes relative to macrophages; and (ii) increased intra-articular residence time relative to a corresponding nanoparticle lacking the biomembrane component.

In a second aspect, the disclosure provides the hybrid nanoparticle of the first aspect, in which the SNARE protein comprises VAMP-3.

In a third aspect, the disclosure provides the hybrid nanoparticle of the first or second aspect, in which the collagen-binding targeting moiety comprises a collagen-II antibody.

In a fourth aspect, the disclosure provides the hybrid nanoparticle of any of the first through third aspects, in which the senolytic agent comprises a combination of dasatinib and quercetin.

In a fifth aspect, the disclosure provides the hybrid nanoparticle of any of the first through fourth aspects, in which the therapeutic payload further comprises a sirtuin activator.

In a sixth aspect, the disclosure provides the hybrid nanoparticle of the fifth aspect, in which the sirtuin activator comprises MDL-800.

In a seventh aspect, the disclosure provides the hybrid nanoparticle of any of the first through sixth aspects, in which the hybrid nanoparticle has (a) an average diameter of 100–200 nm; and (b) a polydispersity index of less than 0.3.

In an eighth aspect, the disclosure provides the hybrid nanoparticle of any of the first through seventh aspects, in which the biomembrane component further comprises cadherins.

In a ninth aspect, the disclosure provides the hybrid nanoparticle of the method of any of the first through eighth aspects, in which the PEGylated phospholipid comprises DSPE-PEG-2000.

In a tenth aspect, the disclosure provides the hybrid nanoparticle of any of the first through ninth aspects, in the nanoparticle is unilamellar.

In an eleventh aspect, the disclosure provides a method of making a hybrid nanoparticle. The method of making a hybrid nanoparticle comprises (a) extracting a biomembrane from chondrocytes, the biomembrane comprising SNARE proteins and CD47; (b) combining the biomembrane with a synthetic lipid mixture comprising phosphatidylcholine, cholesterol, and a PEGylated maleimide lipid; (c) extruding the combination through a membrane having a pore size of about 100 nm to form hybrid nanoparticles; and (d) conjugating a collagen-binding antibody to the hybrid nanoparticles via thiol–maleimide chemistry.

In a twelfth aspect, the disclosure provides method of treating osteoarthritis in a subject. The method of treating osteoarthritis in a subject comprises administering intra-articularly to the subject the hybrid nanoparticle of any of the first through tenth aspects in an amount effective to: (a) selectively eliminate senescent chondrocytes; (b) reduce inflammatory signaling associated with senescence-associated secretory phenotype (SASP); and (c) inhibit progression of cartilage degeneration.

In a thirteenth aspect, the disclosure provides the method of the twelfth aspect, in which the hybrid nanoparticle selectively delivers the senolytic agent to cartilage while avoiding uptake by macrophages.

In a fourteenth aspect, the disclosure provides the method of the twelfth or thirteenth aspect, in which the hybrid nanoparticle is retained within a joint space at the location of the intra-articular administration to provide sustained delivery of the senolytic agent to cartilage within the joint space.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modification and variations come within the scope of the appended claims and their equivalents.

As used throughout, the singular forms “a,” “an” and “the” include plural referents unless the context clearly dictates otherwise. Thus, for example, reference to “a” component includes aspects having two or more such components, unless the context clearly indicates otherwise.

Optional or optionally means that the subsequently described event or circumstances may or may not occur. The description includes instances where the event or circumstance occurs and instances where it does not occur.

It should be understood that any two quantitative values assigned to a property or measurement may constitute a range of that property or measurement, and all combinations of ranges formed from all stated quantitative values of a given property or measurement are contemplated in this disclosure.

As used here and in the appended claims, the words "comprise," "has," and "include" and all grammatical variations thereof are each intended to have an open, non-limiting meaning that does not exclude additional elements or steps.

Having described the subject matter of the present disclosure in detail and by reference to specific embodiments thereof, it is noted that the various details disclosed within should not be taken to imply that these details relate to elements that are essential components of the various embodiments described within, even in cases where a particular element is illustrated in each of the drawings that accompany the present description. Further, it should be apparent that modifications and variations are possible without departing from the scope of the present disclosure, including, but not limited to, embodiments defined in the appended claims. More specifically, although some aspects of the present disclosure are identified as particularly advantageous, it is contemplated that the present disclosure is not necessarily limited to these aspects.

What is claimed is:

1. A hybrid nanoparticle comprising:
(a) a synthetic lipid bilayer comprising phosphatidylcholine, cholesterol, and a PEGylated phospholipid including a maleimide-functionalized PEG lipid;
(b) a biomembrane component derived from chondrocytes and incorporated into the synthetic lipid bilayer, the biomembrane component comprising:
(i) at least one SNARE protein configured to promote homotypic membrane fusion with chondrocytes; and
(ii) CD47 configured to inhibit phagocytic uptake;
(c) a collagen-binding targeting moiety covalently conjugated to the nanoparticle via the maleimide-functionalized PEG lipid; and
(d) a therapeutic payload comprising a senolytic agent,
wherein the hybrid nanoparticle exhibits:
(i) preferential internalization into chondrocytes relative to macrophages; and
(ii) increased intra-articular residence time relative to a corresponding nanoparticle lacking the biomembrane component.

2. The hybrid nanoparticle of claim 1, wherein the SNARE protein comprises VAMP-3.

3. The hybrid nanoparticle of claim 1, wherein the collagen-binding targeting moiety comprises a collagen-II antibody.

4. The hybrid nanoparticle of claim 1, wherein the senolytic agent comprises a combination of dasatinib and quercetin.

5. The hybrid nanoparticle of claim 1, wherein the therapeutic payload further comprises a sirtuin activator.

6. The hybrid nanoparticle of claim 5, wherein the sirtuin activator comprises MDL-800.

7. The hybrid nanoparticle of claim 1, wherein the nanoparticle has:
(a) an average diameter of 100–200 nm; and
(b) a polydispersity index of less than 0.3.

8. The hybrid nanoparticle of claim 1, wherein the biomembrane component further comprises cadherins.

9. The hybrid nanoparticle of claim 1, wherein the PEGylated phospholipid comprises DSPE-PEG-2000.

10. The hybrid nanoparticle of claim 1, wherein the nanoparticle is unilamellar.

11. A method of making a hybrid nanoparticle, comprising:
(a) extracting a biomembrane from chondrocytes, the biomembrane comprising SNARE proteins and CD47;
(b) combining the biomembrane with a synthetic lipid mixture comprising phosphatidylcholine, cholesterol, and a PEGylated maleimide lipid;
(c) extruding the combination through a membrane having a pore size of about 100 nm to form hybrid nanoparticles; and
(d) conjugating a collagen-binding antibody to the hybrid nanoparticles via thiol–maleimide chemistry.

12. A method of treating osteoarthritis in a subject, comprising:
administering intra-articularly to the subject the hybrid nanoparticle of claim 1 in an amount effective to:
(a) selectively eliminate senescent chondrocytes;
(b) reduce inflammatory signaling associated with senescence-associated secretory phenotype (SASP); and
(c) inhibit progression of cartilage degeneration.

13. The method of claim 12, wherein the hybrid nanoparticle selectively delivers the senolytic agent to cartilage while avoiding uptake by macrophages.

14. The method of claim 12, wherein the hybrid nanoparticle is retained within a joint space at the location of the intra-articular administration to provide sustained delivery of the senolytic agent to cartilage within the joint space.
